(19) United States
(12) Patent Application Publication
(10) Pub. No.: US 2010/0092454 A1
(43) Pub. Date: Apr. 15, 2010

(54) NOVEL USE OF ANTIDEPRESSANT COMPOUNDS AND RELATED COMPOSITIONS

(76) Inventors: Enrico De Vivo, Venaria Reale (IT); Antonio Ponzetto, Moncalieri (IT); Chiara Cesano, Torino (IT); Luisa Gennero, Torino (IT); Gianfranco Merizzi, Torino (IT); Emanuella Morra, Torino (IT)

Correspondence Address: Steinfl & Bruno, 301 N Lake Ave Ste 810, Pasadena, CA, 91101, US

(21) Appl. No.: 12/520,503

(22) PCT Filed: Dec. 20, 2007
(86) PCT No.: PCT/IT2007/000891

§ 371 Date: Jun. 19, 2009

(30) Foreign Application Priority Data
Dec. 22, 2006 (IT) TO2006A000918
Dec. 22, 2006 (IT) TO2006A000920
Aug. 20, 2007 (IT) TO2007A000603

(51) Int. Cl. A61K 38/43; A61K 38/46; A61K 38/48; A61K 36/38; A61K 31/55; A61K 31/4525; A61K 31/15; A61K 31/138; A61K 31/135

(52) U.S. Cl. 424/94.65; 424/94.1; 424/94.6; 424/94.63; 424/94.67; 424/725; 514/217; 514/338; 514/640; 514/651; 514/654

(57) ABSTRACT

What is described is a use of an antidepressant compound, preferably belonging to the class of selective serotonin reuptake inhibitors (SSRI), for regenerating the integumentary system and/or for stimulating the growth, the original trophism and/or original pigmentation of the corresponding cutaneous appendages, particularly body hair and/or head hair, in mammals. For this purpose, the antidepressant can be formulated in a cosmetic preparation, a pharmaceutical composition, a medical device, or in the form of a culture medium, alone or in combination with a further active ingredient such as a proteolytic enzyme and/or a vitamin.

The present invention relates to a novel use of antidepressant compounds.

More specifically, the invention relates to the use of an antidepressant compound, preferably belonging to the class of selective serotonin reuptake inhibitors (SSRI), in medical or cosmetic applications which require the regeneration of skin tissue and/or the stimulation of the growth of the integumentary system and of the corresponding cutaneous appendages such as body hair and head hair and/or the recovery of the original pigmentation and/or trophism of the aforesaid cutaneous appendages, including stimulation of the vitality of the hair follicles.

### TECHNICAL BACKGROUND OF THE INVENTION

The Skin

In histological terms, the cutaneous tissue, also called skin or cuticle, is composed of three layers, namely the epidermis, the dermis and the hypodermis.

The epidermis is the outermost layer of the skin, and is formed by numerous clearly distinguished cell layers. The outermost of these is called the stratum corneum; it is composed of anucleate residues which have lost most of the cellular water, and it is hardened; this is caused by the presence of keratin, a protein synthesized in large quantities by specialized tissue cells called keratinocytes. The surface layer is therefore composed of stratified lamellae of cell residues, which continually desquamate and must be replaced throughout the individual's life.

The following layers can be distinguished in the epidermis:
the stratum corneum (apoptotic cells which have been reduced to plates and Merkel cells with afferent nerve endings);
the stratum lucidum, having a lamellar structure and also composed of anucleate residues;
the stratum granulosum (keratin-rich squamous cells);
the stratum spinosum (polyhedral cells in which there is a progressive accumulation of membrane proteins and lamellar granules and ramified Langerhans cells with defensive functions); and
a basal layer (keratinocytes having cubic cells joined together by desmosomes, and melanocytes which produce melanin).

The basal layer contains the numerous stem cells which give rise to new keratinocytes, enabling the tissue to be regenerated throughout a lifetime. The basal layer is in direct contact with the dermis through the basal membrane. The epidermis acts as a barrier against external physical and chemical agents (such as heat, cold, solar radiation, chemical substances present in the environment or secreted by plants or animals) and against pathogens such as bacteria, fungi and the like.

The dermis is divided into the papillary, median and deep regions. Numerous blood and lymph vessels run into the dermis, and various nerve structures and the cutaneous appendages are present. The important appendages are the hair follicles, the sebaceous glands and the sweat glands. The cells present in the dermis are of various types, the important ones being fibrocytes, histiocytes, mast cells and melanocytes.

The hypodermis or subcutaneous layer is composed of a loose connective tissue which delimits spaces containing numerous adipocytes.

Wrinkles are essentially caused by facial expressions, as a result of a reflex action of the facial muscles, or by ageing, as a result of the irreversible slackening of the skin.

Expression wrinkles also appear on the faces of young people, due to a reflex action of the facial muscles. These expression wrinkles or “creases” are different from those due to ageing, because they appear on an epidermis which has not lost its normal elasticity. Some particularly emotional persons have many creases or wrinkles on their faces, which from time to time, and often involuntarily, reflect all the emotions felt by these persons.

On the other hand, wrinkles due to ageing are caused by the slackening of the skin, the reduction of the quantity of intra- and intercellular liquid and the qualitative and quantitative modification of the cellular fatty acids. The phenomenon begins to appear from puberty, but only becomes appreciable at around 30 years, with the appearance of the first wrinkles at the sides of the mouth, under the eyelids and on the forehead.

The processes taking place in the skin, from the dermis to the epidermis, and from the microvascular system to the sebaceous/follicle and pigmentary systems, gradually lead to the maturity and subsequently the ageing of the skin, and are inevitable and irreversible.

The skin picture comprising wrinkles, crows' feet, and drying and slackening of the skin is the expression of the normal organic deterioration which occurs with the passage of time. The menopause and its characteristic endocrine disruption have a considerable effect on the development of these aesthetically displeasing phenomena. It is known that age-related lipid peroxidation processes and internal disturbances can also affect and aggravate the skin condition.

The ageing of the epidermis leads to modifications of cell reproduction, typically in the basal layer of the skin, and a decrease in the layer of Malpighian cells which are extremely important for the epidermis.

Some substances, such as phospholipids used in the form of liposomes, have been found to be useful for maintaining the turgidity of the epidermal cells. With the passage of time, the stratum corneum appears shrivelled, rough and dehydrated, and therefore it is useful to treat it with vitamin-type substances with natural moisturizing and other agents, which limit and retard the appearance of these displeasing phenomena. Correctly formulated skin care products therefore act as preventive and helpful agents for counteracting the appearance of wrinkles, even if they cannot rejuvenate the skin. The primary purpose is to maintain the moisture level, and to prevent the loss of elasticity and dehydration typical of ageing skins which tend to be lacking in water. However, they must not form a barrier which prevents the normal transpiration of the skin.

In order to prevent or retard the processes of skin ageing, it is first necessary to counteract the inevitable modification of the mucopolysaccharide component of the fundamental substance and the slackening of the elastic and collagen component of the dermis.

A valid form of prevention comprises the retardation of the flattening of the papillary projections, the prevention of the slowing of the blood circulation, the avoidance of an increase in non-removed toxins, and the attempt to avoid a poor supply of nutrition to the tissues above the papillary region of the dermis.

Correctly formulated skin care products therefore act as preventive and helpful agents for counteracting the appearance of wrinkles, although it is true that they cannot rejuvenate the skin. The primary purpose is to maintain the moisture level, and to prevent the loss of elasticity and dehydration typical of ageing skins which tend to be lacking in water. However, they must not form a barrier which prevents the normal transpiration of the skin.

The Scalp

Numerous studies have been carried out on the growth of hair in general and of head hair in particular, and there have also been numerous attempts to develop compositions for overcoming the problems of alopecia, effluvium and defluvium in mammals, including humans.

In this context, it is worth citing the study by M. Robinson et al. [6] on the in vitro development of the follicles of adult rat vibrissae. This study laid the groundwork for the definition of a culture protocol for keeping the hair follicles alive for more than 20 days (before this research, the in vitro growth of hairs generally ceased prematurely, unlike the growth in vivo [6]). Microscopic examination showed that, in spite of widespread pathological changes in the epithelium of the follicle, the cells of the follicle showed a considerable capacity for recovery. Thus these data confirmed that hair loss was not attributable to the loss of the regenerative capacity of the follicles (which still remained alive, in a state of quiescence, or rather of reversible atrophy), but to a number of factors which affected the life cycle of the follicle [1-6].

The term “alopecia” denotes the absence or deficiency of body or head hair in the skin areas in which it is normally present. The term “alopecia” covers both hypotrichosis, signifying a deficiency of body or head hair, and baldness, signifying the irreversible loss of head hair.

The term “defluvium”, on the other hand, is used to denote a loss of head hair which is abnormal in quantity and quality, while the term “effluvium” is used to refer to cases in which the loss is numerically very high, up to many hundreds of hairs per day, and qualitatively homogeneous.

Alopecia has conventionally been divided into temporary forms (a transient functional inhibition of the hair papilla) and permanent forms (disappearance of the follicle and of the germinative papilla). These are to be distinguished from pseudo-alopecias, in which the hairs have been torn out or have broken up (trichoclasia) as a result of traumatic, chemical, or infective events, or due to congenital abnormalities of the shaft.

Alopecia can arise as a result of genetic factors, ageing, or local or systemic diseases. Seborrhoeic dermatitis and psoriasis are the pathologies that most commonly affect the scalp, but they rarely lead to alopecia. Alopecia can be of the cicatricial or non-cicatricial, toxic or drug-induced, areata or pseudopelade of Brocq, iatrogenic (generally due to medicines), post-pregnancy, or post-infective type, and it can also be caused by trichotillomania, ringworm, kerion and crusted ringworm. Alopecia can also be caused by lupus erythematosus (in both the systemic and the fixed discoid form), scleroderma, lichen planus, follicular mucinosis or folliculitis decalvans, and by aplasia cutis or tumours [1-4].

Androgenic alopecia does not appear if the concentration of male hormones does not reach the levels present in adults, and therefore it never appears before puberty. In humans, baldness is not due to an excess of androgenic hormones, but to an excessive response of the integumentary system to these hormones [5].

The sensitivity of hairs, or rather of the hair follicles, to androgenic hormones depends mainly on an enzyme, namely type 2 5-alpha-reductase, produced by the cells of the follicle [5]. This enzyme converts testosterone, the principal male hormone, into its most powerful derivative, namely dihydrotestosterone or DHT, which is mainly responsible for androgenic alopecia. The follicles of the areas of the scalp which are subject to baldness produce large quantities of this enzyme, and therefore large quantities of DHT [5].

Androgenic baldness in women starts at around 35 years and is typically manifested in three stages. In young women especially, thinning is frequently more evident above the forehead [4-5]. In menopausal women, however, thinning at the temples, similar to that which occurs in men, is frequently observed. However, even in the most severe cases, complete baldness is never observed, but only a considerable thinning [4-5]. In women, androgenic baldness can be caused by an excess of male hormones or by excessive sensitivity of the integumentary system to completely normal levels of androgen [4-5].

### DESCRIPTION OF THE INVENTION

The object of the present invention is to find a valid and efficient solution for stimulating and improving the vitality and trophism of the whole integumentary system, including the skin tissue, the scalp and the corresponding cutaneous appendages, such as body hair and head hair, thus inducing its regeneration.

According to the present invention, this object is achieved by means of the solution claimed specifically in the following claims. The claims form an integral part of the technical teachings provided herein in relation to the invention.

The invention is based on the observation of a specific proliferative stimulus imparted to the skin, the scalp and the corresponding cutaneous appendages (body hair and head hair) by antidepressant compounds, especially those belonging to the class of selective serotonin reuptake inhibitors (SSRI). This activity is induced by interaction with local and regional receptors of these substances, capable of inducing a recovery of the trophism of ageing skin tissue and of the pathological scalp [14, 15].

This activity of the antidepressant compounds can be usefully applied in the cosmetic and medical fields.

In the cosmetic field, an antidepressant compound can be used for the cosmetic treatment of wrinkles, for recovering the original pigmentation of the cutaneous appendages (body hair and head hair) in mammals, including humans, and for stimulating the growth of the cutaneous appendages (body hair and head hair) in mammals.

In the medical field, the antidepressant compound can be used for regenerating the skin tissue and the scalp in patients who have suffered damage to these tissues, or for treating alopecia, effluvium or defluvium.

Preferably, the antidepressant compound is a selective serotonin reuptake inhibitor (SSRI), a precursor thereof, or a natural or synthetic derivative thereof. Among SSRIs, paroxetine is preferred.

For the medical and cosmetic purposes mentioned above and in the appended claims, the antidepressant compound can be prepared in the form of a cosmetic composition, a pharmaceutical composition, a medical device, or a culture medium for regenerating the skin tissue in vitro or, for stimulating the growth, nutrition and/or original pigmentation of the cutaneous appendages (body hair and/or head hair) in vitro. These preparations, comprising the antidepressant compound as the active principle, can also optionally comprise one or more further synergistic active ingredients such as proteolytic enzymes and/or vitamins. These preparations can also optionally comprise physiologically acceptable solvents and/or diluents, as well as the usual excipients and/or additives for pharmaceutical or cosmetic compositions.

As mentioned above, the antidepressant compound preferred for use in the scope of the present invention is paroxetine, a well-known medicine belonging to the class of selective serotonin reuptake inhibitors (SSRI). However, for the purpose of the invention, other natural or synthetic antidepressant compounds are also suitable, such as hypericum (obtained from the herb Hypericum perforatum), fluoxetine, fluvoxamine, amitriptyline, desipramine, chlorimipramine, imipramine, nortriptyline and venlafaxine.

In general, the antidepressant compound is used in an amount in the range from 100 mg/kg to 100 g/kg, preferably 0.05 g/kg to 20 g/kg, or even more preferably 0.5 g/kg to 10 g/kg for substantially solid compositions, and in the range from 100 mg/l to 100 g/l, preferably 0.05 g/l to 20 g/l, or even more preferably 0.05 g/l to 10 g/l for substantially liquid compositions.

The proteolytic enzymes which can optionally be used in combination with the antidepressant include, for example, protease, peptidase, papain, papain FU, collagenase (preferably type Ia, type II or type IV), serratiopeptidase, heparanase, DNase, elastase, bromelain, bradykinase, Clostridium peptidase, enzymes expressed by Lactobacillus acidophilus, enzymes expressed by the Aspergillus genus, alliinase, and fibrinolysin. The preferred enzymes are proteases, which are capable of activating three extremely important phenomena which can produce a synergistic effect with the activity of the antidepressant compound, namely:
activation of the growth factors present in the skin;
accelerated absorption of the nutrients present in the preparation;
deep exfoliation of the integumentary systems.

In general, the proteolytic enzymes can be used in an amount in the range from 1 mg/kg to 1 g/kg, or preferably 10 mg/kg to 100 mg/kg, for substantially solid compositions, and in the range from 1 mg/l to 1 g/l, or preferably 10 mg/l to 100 mg/l, for substantially liquid compositions.

Examples of vitamins which can be used in combination with the antidepressant compound and also with the proteolytic enzyme if necessary are retinaldehyde (retinoid), retinoic acid, and their natural or synthetic precursors and derivatives. Retinaldehyde is preferred since it also has a synergistic effect, being capable of inducing rapid tissue regeneration.

In general, the vitamin can be used in an amount in the range from 0.001 mg/kg to 10 g/kg, or preferably 0.01 mg/kg to 1 g/kg, for substantially solid compositions, and in the range from 0.001 mg/l to 10 g/l, or preferably 0.01 mg/l to 1 g/l, for substantially liquid compositions.

The antidepressant compound, optionally combined with the proteolytic enzyme and/or the vitamin, can be formulated in solid or liquid preparations which may be anhydrous or aqueous, for example creams, ointments, pomades, powders, plasters, impregnated membranes, solutions, emulsions, suspensions, vesicular dispersions, lotions, gels or sprays.

The person skilled in the art will be able to prepare these preparations, using the appropriate additives, excipients and/or diluents or vehicles.

In general, a base cream is used as the diluent or vehicle of substantially solid preparations (such as creams, ointments and pomades), while a physiological solution is used as the diluent or vehicle of substantially liquid preparations.

Examples of the additional ingredients which can be used in addition to the principal active ingredients are:
vitamins and vitamin factors: retinoic acid, retinol, alpha-tocopherol, tocopheryl acetate, beta-carotene, ascorbic acid, pantothenic acid, D-calcium pantothenate, pyridoxine, pyridoxine HCl, folic acid, niacinamide (Nicotinamide), riboflavin, cobalamine, para-aminobenzoic acid, and biotin, and the vitamin factors para-aminobenzoic acid (PAB), inositol and myo-inositol;
glucosaminoglycans: hyaluronic acid, chondroitin sulphates;
saccharides: rice starch, glucose, sucrose, glucans, mannans, glucomannans, fucose, fructose, heparan sulphates, pectins, starches and their alcohol derivatives;
triterpene acids or heterosides: madecassoside, asiaticoside and their derivatives and precursors;
peptides: glutathione, collagen, elastin, wheat extract;
supplements: royal jelly, pyruvate (for example sodium pyruvate), plant extracts such as Centella asiatica or Hypericum perforatum;
corticosteroids: such as dexamethasone (for example dexamethasone 21-phosphate disodium);
anticholinergics: such as scopolamine and scopolamine butylbromide;
essential and non-essential amino acids.

The invention will now be described in detail, purely by way of a non-limiting example, with reference to some preferred embodiments.

These embodiments are represented by the composition designated as Ru-BASE described below, which contains paroxetine as the main active ingredient. The Ru-BASE composition was prepared as a cream and as an infusion.

Cream, gel and infusion compositions, comprising the antidepressant alone as the active ingredient, in a physiologically acceptable medium (such as a base cream or a physiological solution), are listed below as Ru-BASE-CREMA, Ru-BASE-GEL and Ru-BASE-INFUS, and are illustrated in Tables 1, 2 and 3 respectively. The subsequent Tables 4 to 18 illustrate compositions of the Ru-BASE type in which the paroxetine antidepressant compound is combined with one or more additional active ingredients.

In particular, Tables 4, 5 and 6 illustrate, respectively, Ru-BASE compositions in a cream, gel and infusion form, comprising a combination of paroxetine and protease enzyme. These compositions are designated as Ru-BASE-CREMA-PROTEO-PLUS, Ru-BASE-GEL-PROTEO-PLUS and Ru-BASE-INFUS-PROTEO-PLUS, respectively.

Tables 7, 8 and 9 illustrate, respectively, Ru-BASE compositions in a cream, gel and infusion form, comprising a combination of paroxetine and retinaldehyde. These compositions are designated as Ru-BASE-CREMA-RET-PLUS, Ru-BASE-GEL-RET-PLUS and Ru-BASE-INFUS-RET-PLUS, respectively.

Tables 10, 11 and 12 illustrate, respectively, Ru-BASE compositions in a cream, gel and infusion form, comprising a combination of paroxetine and retinoic acid as an alternative to retinaldehyde. These compositions are designated as Ru-BASE-CREMA-aRET-PLUS, Ru-BASE-GEL-aRET-PLUS and Ru-BASE-INFUS-aRET-PLUS, respectively.

Tables 13, 14 and 15 illustrate, respectively, Ru-BASE compositions in a cream, gel and infusion form, comprising a combination of paroxetine, protease enzyme and retinaldehyde. These compositions are designated as Ru-BASE-CREMA-COMBO-PLUS, Ru-BASE-GEL-COMBO-PLUS and Ru-BASE-INFUS-COMBO-PLUS, respectively.

Tables 16, 17 and 18 illustrate, respectively, Ru-BASE compositions in a cream, gel and infusion form, comprising a combination of paroxetine, protease enzyme, and retinoic acid as an alternative to retinaldehyde. These compositions are designated as Ru-BASE-CREMA-COMBO2-PLUS, Ru-BASE-GEL-COMBO2-PLUS and Ru-BASE-INFUS-COMBO2-PLUS, respectively.

The histological results obtained in vivo after six months of treatment with the Ru-BASE compositions illustrated below confirm the re-establishment of trophism of the wrinkles by means of a modulation of the life cycle of the fibroblast with recovery of the atrophic states and prevention of the proliferation of these. The re-establishment of trophism induced by the Ru-BASE compositions is morphologically comparable to the healthy in vivo state with optimal histofunctional characteristics.

These observations led the inventors to test the Ru-BASE on scalp biopsies in vitro (areas affected by alopecia whose nature was not yet determined).

The Ru-BASE-INFUS composition was found to be capable of stimulating hair growth in vitro, improving the vitality of both atrophic and non-atrophic hair follicles. After twenty-one days of treatment in vitro, all the hairs, whether new or not, recovered their original nutrition, appeared reinvigorated, with the original pigmentation, and showed an increased diameter of the shaft, which was healthy and free of desquamation.

The term “base” in the tables showing the cream compositions denotes a base in the form of a cream or emulsion (O/A or A/O) (such as water, white vaseline, cetostearyl alcohol, liquid paraffin, Ceteth-20, sodium phosphate, p-chloro-m-cresol, and phosphoric acid).

The term “gel base” in the tables showing the gel compositions denotes a gel base, such as carbopol or cellulose derivatives.

TABLE 1
Ru-BASE-CREMA composition

| Substance | Concentration |
|---|---|
| Paroxetine | 2.5 g/kg |
| Base | q.s. |

TABLE 2
Ru-BASE-GEL composition

| Substance | Concentration |
|---|---|
| Paroxetine | 2.5 g/L |
| gel base | q.s. per kg of product |

TABLE 3
Ru-BASE-INFUS composition

| Substance | Concentration |
|---|---|
| Paroxetine | 2.5 g/L |
| Physiological solution | q.s. per 1 L of product |

TABLE 4
Ru-BASE-CREMA-PROTEO-PLUS composition

| Substance | Concentration | |
|---|---|---|
| Paroxetine | 2.5 | g/kg |
| Papain FU | 22 | mg/kg |
| or protease | | |

| | |
|---|---|
| base | q.s. |

TABLE 5
Ru-BASE-GEL-PROTEO-PLUS composition

| Substance | Concentration | |
|---|---|---|
| Paroxetine | 2.5 | g/L |
| Papain FU | 22 | mg/L |
| or protease | | |

| | |
|---|---|
| gel base | q.s. per kg of product |

TABLE 6
Ru-BASE-INFUS-PROTEO-PLUS composition

| | |
|---|---|
| Substance | Concentration |

| | | |
|---|---|---|
| Paroxetine | 2.5 | g/L |
| Papain FU | 22 | mg/L |
| or protease | | |

| | |
|---|---|
| Physiological solution | q.s. per 1 L of product |

TABLE 7
Ru-BASE-CREMA-RET-PLUS composition

| Substance | Concentration | |
|---|---|---|
| Paroxetine | 2.5 | g/kg |
| Retinaldehyde | 500 | mg/kg |

| | |
|---|---|
| base | q.s. |

TABLE 8
Ru-BASE-GEL-RET-PLUS composition

| Substance | Concentration | |
|---|---|---|
| Paroxetine | 2.5 | g/L |
| Retinaldehyde | 500 | mg/L |

| | |
|---|---|
| gel base | q.s. per kg of product |

TABLE 9
Ru-BASE-INFUS-RET-PLUS composition

| Substance | Concentration | |
|---|---|---|
| Paroxetine | 2.5 | g/L |
| Retinaldehyde | 500 | mg/L |

| | |
|---|---|
| Physiological solution | q.s. per 1 L of product |

TABLE 10
Ru-BASE-CREMA-aRET-PLUS composition

| Substance | Concentration | |
|---|---|---|
| Paroxetine | 2.5 | g/kg |
| Retinoic acid | 0.02 | mg/kg |

| | |
|---|---|
| base | q.s. |

TABLE 11
Ru-BASE-GEL-aRET-PLUS composition

| Substance | Concentration | |
|---|---|---|
| Paroxetine | 2.5 | g/L |
| Retinoic acid | 0.02 | mg/kg |

| | |
|---|---|
| gel base | q.s. per kg of product |

TABLE 12
Ru-BASE-INFUS-aRET-PLUS composition

| Substance | Concentration | |
|---|---|---|
| Paroxetine | 2.5 | g/L |
| Retinoic acid | 0.02 | mg/kg |

| | |
|---|---|
| Physiological solution | q.s. per 1 L of product |

TABLE 13
Ru-BASE-CREMA-COMBO-PLUS composition

| Substance | Concentration | |
|---|---|---|
| Paroxetine | 2.5 | g/kg |
| Retinaldehyde | 500 | mg/kg |
| Protease | 22 | mg/kg |

| | |
|---|---|
| base | q.s. |

TABLE 14
Ru-BASE-GEL-COMBO-PLUS composition

| Substance | Concentration | |
|---|---|---|
| Paroxetine | 2.5 | g/L |
| Retinaldehyde | 500 | mg/L |
| Protease | 22 | mg/L |

| | |
|---|---|
| gel base | q.s. per kg of product |

TABLE 15
Ru-BASE-INFUS-COMBO-PLUS composition

| Substance | Concentration | |
|---|---|---|
| Paroxetine | 2.5 | g/L |
| Retinaldehyde | 500 | mg/L |
| Protease | 22 | mg/L |

| | |
|---|---|
| Physiological solution | q.s. per 1 L of product |

TABLE 16
Ru-BASE-CREMA-COMBO2-PLUS composition

| Substance | Concentration | |
|---|---|---|
| Paroxetine | 2.5 | g/kg |
| Retinoic acid | 0.02 | mg/kg |
| Protease | 22 | mg/kg |

| | |
|---|---|
| base | q.s. |

TABLE 17
Ru-BASE-GEL-COMBO2-PLUS composition

| Substance | Concentration | |
|---|---|---|
| Paroxetine | 2.5 | g/L |
| Retinoic acid | 0.02 | mg/kg |
| Protease | 22 | mg/L |

| | |
|---|---|
| gel base | q.s. per kg of product |

TABLE 18
Ru-BASE-INFUS-COMBO2-PLUS composition

| Substance | Concentration | |
|---|---|---|
| Paroxetine | 2.5 | g/L |
| Retinoic acid | 0.02 | mg/kg |
| Protease | 22 | mg/L |

| | |
|---|---|
| Physiological solution | q.s. per 1 L of product |

All the biopsy tissue samples responded positively to the use of the Ru-BASE compositions, remaining vital, depositing collagen and having an ordered and three-dimensional distribution during 6 months of culture in vitro. The vitality of the atrophic epithelial tissues appeared to be markedly improved after 15 days of treatment.

All the test samples of patients with male, female and cicatricial alopecia remained vital in culture, with the hair follicles active for six months. All the treated biopsies showed a marked and constant re-thickening of the rows of hairs, always determined by a topographic order of distribution and an underlying tissue which was well nourished and free of lesions.

Without wishing to adopt any specific theory on this matter, the present inventors consider that the results obtained with the Ru-BASE compositions have indicated that the state of cutaneous atrophy, and more generally integumentary atrophy, which occurs in degenerative processes is reversible. Indeed, the Ru-BASE compositions have been shown to be capable of inducing excellent growth and development of skin tissue, and of inducing the regrowth of hair in vitro with normal histofunctional characteristics.

The experiments which were conducted are described more fully in the following section.

Biopsies and Prototype Solutions

All the samples (biopsies of cartilage tissue) were washed three times with physiological solution and antibiotics (100 units/ml penicillin+100 μg/ml streptomycin+160 mg/L gentamicin) for 10 minutes at ambient temperature.

The biopsies were then divided into three parts (two controls and one sample for each patient). The sample was treated with a Ru-BASE solution with a final concentration of 1× in 15 cm plates (Lab-Tek Chamber Slides, made by Nunc, Kamstrup, Denmark).

Two types of controls were prepared, namely a negative control (1) treated solely with physiological solution and antibiotics (as described above), and a negative control (2) treated with ordinary cell culture media.

1. The control biopsy specimens were suspended in physiological solution in 15 cm plates (Lab-Tek Chamber Slides, Nunc, Kamstrup, Denmark).

2. The control biopsy specimens were then placed in 15 cm plates (Lab-Tek Chamber Slides, Nunc, Kamstrup, Denmark) in RPMI 1640 medium supplemented with: 10% FBS (Celbio, Milan, Italy)

100 units/ml penicillin

100 μg/ml streptomycin

160 mg/L gentamicin (Schering-Plough, Milan, Italy)

2 mM L-glutamine (Life Technologies; Growth Medium).

All the samples were placed in a Heraeus incubator which was thermostatically maintained at a temperature of 37° C. with an atmosphere containing 8% of constantly supplied CO₂ (v/v in air).

Skin Biopsies

Matrix Staining Protocol

After washing three times for 10 minutes at ambient temperature in PBS (pH 7.4), the samples were resuspended in a 4% fixing solution of paraformaldehyde in D-MEM (Gibco) at pH 7.4 for one hour at ambient temperature. All the biopsies used for the study were treated with Alcian blue. This dye is composed of a group of water-soluble polyvalent basic dyes. The blue colour is due to the presence of copper in the molecule.

Alcian blue in solution with PBS (pH 7.4) at 1% of final concentration p/V was added to a 3% acetic acid solution (pH 2.5). After incubation for two hours at ambient temperature, this composition dyes indelibly, by binding the acid mucopolysaccharides and sulphonated and carboxylated glycoproteins. Specific controls were prepared for each sample.

All the samples were washed three times with PBS (pH 7.4) at ambient temperature for five minutes and were then examined with an optical microscope. A marked increase in type 2 collagen, type 3 collagen and type 4 collagen, which were dyed blue, was noted in the samples treated with the F-BASE composition in the form of a solution, with respect to control 1 and control 2 [13].

Skin Biopsies: Results

Dyeing By the Aldan Blue Colorimetric Method
Control 1 treated with physiological solution: very slight background dyeing, hardly perceptible (score=±).
Control 2 treated with ordinary D-MEM culture medium for biopsies, 10% FBS added, as described above. A very slight diffuse pale blue background dyeing (Alcian blue) was noted (score=++).
Sample treated with F-BASE INFUS. It was noted that the cells, where collagen redeposition had been induced, were clearly dyed with Alcian blue, growing in superimposed layers (score=+++++).

Western Blot

The samples were subjected to phenotype analysis by the Western blot for anti-collagen type II markers (Santa Cruz Biotechnology, America, California), anti-collagen type III (Santa Cruz Biotechnology, America, California), anti-collagen type IV (Santa Cruz Biotechnology, America, California), and anti-aggrecan (Santa Cruz Biotechnology, America, California). After five washes, the membrane were incubated with the corresponding secondary antibodies (1:1000) conjugated with horse radish peroxidase (HRP, SantaCruz Biotechnologies Inc., Santa Cruz, Calif., USA) for one hour at ambient temperature, as shown in Table 19 below.

Characterization of the Skin Tissue Treated With Ru-BASE By Comparison With Untreated Controls

The results relating to the expression of type II collagen, type III collagen, type IV collagen and aggrecan were expressed on a quantitative scale as shown below:

TABLE 19

| Markers | Control 1 | Control 2 | Sample |
|---|---|---|---|
| type II collagen | −/+ | ++ | +++++ |
| type III collagen | −/+ | + | +++++ |
| type IV collagen | −/+ | ++ | +++++ |
| aggrecan | −−− | −−− | ++++ |

Key
−−− = no band
−/+ = slight presence of band
+ = thin band present
++ = medium band present
+++ = extended band present
++++ = large band present
+++++ = widespread band present

Scalp Biopsies

Immunofluorescence Protocol

After washing three times for 10 minutes at ambient temperature in PBS (pH 7.4), the samples were resuspended in a 4% fixing solution of paraformaldehyde in RPMI 1640 at pH 7.4 for one hour at ambient temperature. After embedding in paraffin, the samples were sectioned and placed on slides. The sections were dyed with haematoxylin-eosin, anti-cytokeratin 10 monoclonal antibodies (Santa Cruz Biotechnology, America, California), and anti-cytokeratin 11 monoclonal antibodies (Santa Cruz Biotechnology, America, California). Specific controls were prepared for each monoclonal antibody with the corresponding isotypes (Santa Cruz Biotechnology, America, California). All the samples placed on slides were examined with an optical microscope after being sealed with Moviol and cover slides.

Western Blot for Cytokeratin

The biopsies, suspended in a lysis buffer (1% SDS, 30 mM Tris pH 6.8, 5% glycerol) to which protease inhibitors were added (Protease Inhibitor Cocktail, Calbiochem, San Diego, Calif.), were homogenized, followed by incubation of the samples for 30 minutes at 4° C. The resulting lysates were centrifuged at 12,000 r.p.m. for 20 minutes at 4° C. and the supernatant was collected; the protein concentration of the samples was evaluated by the Bio-Rad method (Benchmark Plus assay, Bio-Rad). Before the electrophoresis run, the samples were boiled for 5 minutes in the presence of beta-mercaptoethanol and bromophenol blue. The samples were subjected to electrophoresis in a 12% gel (SDS-PAGE) and transferred to a PVDF membrane (Perkin Elmer Inc.). The membranes were saturated with methanol at ambient temperature and then incubated with the following primary antibodies diluted in PBS with 5% skimmed milk powder: anti-cytokeratin 14 with a dilution of 1:500 (SantaCruz Biotechnologies Inc., Santa Cruz, Calif. USA), anti-cytokeratin 18 with a dilution of 1:500 (SantaCruz Biotechnologies Inc., Santa Cruz, Calif. USA) and anti-cytokeratin 19 with a dilution of 1:500 (SantaCruz Biotechnologies Inc., Santa Cruz, Calif. USA) for the whole of one night at 4° C. After five washes, the membranes were incubated with the corresponding secondary antibodies (1:1000) conjugated with horse radish peroxidase (HRP, SantaCruz Biotechnologies Inc., Santa Cruz, Calif., USA) for one hour at ambient temperature. The corresponding bands were displayed with chemiluminescence liquids (Super Signal Western Pico solution, Pierce Biotechnology Inc., Rockford, Ill., USA) and fixed on photographic plates.

Scalp Biopsies: Results

Optical Microscope

These results indicate scalp regrowth with no alopecic pathology, and with a distribution of the normal regrowth stages.

The results relating to the expression of cytokeratin 10 and 11 and the histological dyeing of the biopsy preparations with haematoxylin-eosin (used to display follicular vitality) are shown in the table and are expressed on a quantitative scale.

An analysis of the results in Table 20 shows that, under the optical microscope (with eosin and haematoxylin dyeing), there was a marked increase in the number of follicles in the samples treated with the Ru-BASE composition proposed by the invention, with respect to the untreated controls. Furthermore, all the follicles appeared to be well nourished, vital and active in the treated samples, by comparison with the untreated samples in which atrophy or hypotrophy of the follicle was observed. Finally, there was a marked predominance of cytokeratin 10 and 11, typical of normal integumentary tissues with vital and active follicles, in the samples treated with the Ru-BASE composition proposed by the invention, by comparison with the untreated controls [8].

TABLE 20

| Markers | Control 1 | Control 2 | Sample |
|---|---|---|---|
| Cytokeratin 10 | + | ++ | ++++ |
| Cytokeratin 11 | + | ++ | ++++ |
| Eosin/ | ++ | ++ | ++++ |
| Haematoxylin | | | |

Key
−−−−− = no fluorescence
+ = low fluorescence for optical field
++ = medium fluorescence for optical field
+++ = high fluorescence for optical field
++++ = very high fluorescence for optical field
pz = patient

Western Blot

The samples were subjected to Western blot phenotype analysis for cytokeratin 14, cytokeratin 18 and cytokeratin 19 markers, as shown in Table 21 below.

The results are highly positive for the production of cytokeratin 14, cytokeratin 18, and cytokeratin 19 in the treated samples, and particularly in the scar samples treated for six months in vitro with the Ru-BASE composition proposed by the present invention, by comparison with only a slightly positive result for the production of cytokeratin 14, cytokeratin 18, and cytokeratin 19 in the untreated control samples, as shown in Table 20. Cytokeratin 14, 18 and 19 are expressed in normal integumentary tissues with vital and active follicles during the stages of cell differentiation, hair follicle growth and hair formation control [8-9].

TABLE 21

| Markers | Control 1 | Control 2 | Sample |
|---|---|---|---|
| Cytokeratin 14 | + | + | ++++ |
| Cytokeratin 18 | + | + | ++++ |
| Cytokeratin 19 | + | + | ++++ |

Key
−−− = no band
−/+ = slight presence of band
+ = thin band present
++ = medium band present
+++ = extended band present
++++ = large band present
+++++ = widespread band present
pz = patient

Compositions comprising hypericum (hypericum perforatum) as an alternative antidepressant to paroxetine, in combination with some preferred ancillary substances, are described below by way of a further example. These compositions proved to be effective in the regeneration and re-nutrition of the skin tissue.

TABLE 22
F-BASE-CREMA compositon

| Substance | Concentration, mg/kg | |
|---|---|---|
| Hypericum (Hypericum | 5 | g/kg |
| perforatum) | | |
| Royal jelly | 50 | g/kg |
| Ascorbic acid | 100 | g/kg |
| D-calcium pantothenate | 500 | mg/kg |
| Cobalamine | 0.5 | mg/kg |
| Retinoic acid | 2 | mg/kg |
| Tocopheryl acetate | 2,500 | mg/kg |
| Papain | 22 | mg/kg |
| Reduced glutathione | 510 | mg/kg |
| Madecassoside | 1 | g/kg |

| | |
|---|---|
| base | q.s. |

TABLE 23
F-BASE-GEL composition

| Substance | Conc., mg/kg | |
|---|---|---|
| Hypericum (Hypericum | 5 | g/L |
| perforatum) | | |
| Royal jelly | 50 | g/L |
| Ascorbic acid | 100 | mg/L |
| D-calcium pantothenate | 500 | mg/L |
| Cobalamine | 0.5 | mg/L |
| Retinoic acid | 2 | mg/L |
| Tocopheryl acetate | 2,500 | mg/L |
| Papain | 22 | mg/L |
| Glutathione (reduced) | 510 | mg/L |
| Madecassoside | 1 | g/L |

| | |
|---|---|
| gel base | q.s. per kg of product |

TABLE 24
F-BASE-INFUS composition

| Substance | Conc., mg/kg | |
|---|---|---|
| Hypericum (Hypericum | 5 | g/L |
| perforatum) | | |
| Royal jelly | 50 | g/L |
| Ascorbic acid | 100 | g/L |
| D-calcium pantothenate | 500 | mg/L |
| Cobalamine | 0.5 | mg/L |
| Retinoic acid | 2 | mg/L |
| Tocopheryl acetate | 2,500 | mg/L |
| Papain | 22 | mg/L |
| Glutathione (reduced) | 510 | mg/L |
| Madecassoside | 1 | g/L |

| | |
|---|---|
| Physiological solution | q.s. per 1 L of product |

### REFERENCES

1. Robinson M, Reynolds A J, Gharzi A, Jahoda C A. In vivo induction of hair growth by dermal cells isolated from hair follicles after extended organ culture. J Invest Dermatol. 2001 September; 117(3):596-604.

2. Stem Cells: Scientific Progress and Future Research Directions. Department of Health and Human Services. June 2001.

3. Griffith, L. G. & Naughton, G. Tissue engineering—current challenges and expanding opportunities. Science. 2002; 295:1009-1014.

4. Wagers, A. J., Christensen, J. L., & Weissman, I. L. Cell fate determination from stem cells. Gene Ther. 2002; 9:606-612.

5. Bianco, P. and Cossu, G. Uno, nessuno e centomila: searching for the identity of mesodermal progenitors. Exp Cell Res. 1999 Sep. 15; 251(2):257-63.

6. Rabinovitch, M. & De Stefano, M. J. Cell shape changes induced by cationic anesthetics. J. Exp. Med. 1976; 143: 290-304.

7. Parker F.: “Cute e ormoni” in Williams R. H. eds: “Trattato di Endocrinologia”. 3rd Italian edition, Piccin, Padua. 1979; vol II, ch. 23: 1115-19.

8. Miller E J. A review of biochemical studies on the genetically distinct collagens of skeletal system. Clin Orthop. 1973; 92:260-80.

9. Shapiro F, Koide S, Glimcher M J. Cell origin and differentiation in the repair of full-thickness defects of articular cartilage. J Bone Joint Surg. 1993; 75/A:532-53.

10. Nelea V, Luo L, Demers C N, Antoniou J, Petit A, Lerouge S, R Wertheimer M, Mwale F. Selective inhibition of type X collagen expression in human mesenchymal stem cell differentiation on polymer substrates surface-modified by glow discharge plasma. J Biomed Mater Res A. 2005 Oct. 1; 75(1):216-23.

11. Glowacki J, Yates K E, Maclean R, Mizuno S. In vitro engineering of cartilage: effects of serum substitutes, TGF-beta, and IL-1alpha. Orthod Craniofac Res. 2005 August; 8(3):200-8.

12. Chua K H, Aminuddin B S, Fuzina N H, Ruszymah B H. Insulin-transferrin-selenium prevent human chondrocyte dedifferentiation and promote the formation of high quality tissue engineered human hyaline cartilage. Eur Cell Mater. 2005 Jun. 17; 9:58-67; discussion 67.

13. French M M, Smith S E, Akanbi K, Sanford T, Hecht J, Farach-Carson M C, Carson D D. Expression of the heparan sulfate proteoglycan, perlecan, during mouse embryogenesis and perlecan chondrogenic activity in vitro. J Cell Biol. 1999 May 31; 145(5):1103-15.

14. Kuhn C, Francis R. Gender difference in cocaine-induced HPA axis activation. Neuropsychopharmacology. 1997 June; 16(6):399-407. PMID: 9165495.

15. Czeh B, Muller-Keuker J I, Rygula R, Abumaria N, Hiemke C, Domenici E, Fuchs E. Chronic Social Stress Inhibits Cell Proliferation in the Adult Medial Prefrontal Cortex: Hemispheric Asymmetry and Reversal by Fluoxetine Treatment. Neuropsychopharmacology. 2006 Dec. 13. PMID: 17164819.

1-39. (canceled)

40. A treatment method for a mammal, comprising:
administering to the mammal a composition comprising an effective amount of an antidepressant compound to obtain at least one of:
stimulation of regeneration of the integumentary system of the mammal;
stimulation of at least one of growth, original trophism, and original pigmentation of cutaneous appendages corresponding to the integumentary system of the mammal;
regeneration of damaged skin tissue of the mammal; and
treating of at least one of alopecia, effluvium, and defluvium of the mammal.

41. The method of claim 40, wherein the administering is performed to obtain cosmetic treatment of wrinkles.

42. The method of claim 40, wherein the administering is performed to obtain stimulation of growth of body hair and/or head hair.

43. The method of claim 40, wherein the administering is performed to obtain restoration of original pigmentation of body hair and/or head hair.

44. The method of claim 40, wherein the antidepressant compound is selected from the group consisting of selective serotonin reuptake inhibitors (SSRIs), precursors thereof and natural or synthetic derivatives thereof.

45. The method of claim 44, wherein the antidepressant compound is selected from the group consisting of hypericum, paroxetine, fluoxetine, fluvoxamine, amitriptyline, desipramine, chlorimipramine, imipramine, nortriptyline, venlafaxine, and their precursors and natural or synthetic derivatives.

46. The method of claim 45, wherein the antidepressant compound is paroxetine.

47. The method of claim 40, wherein the administering is performed by administering the antidepressant compound in combination with a proteolytic enzyme.

48. The method of claim 40, wherein the administering is performed by administering the antidepressant compound in combination with a vitamin.

49. The method of claim 40, the administering is performed by administering the antidepressant compound in combination with a proteolytic enzyme and a vitamin.

50. The method of claim 47, wherein the proteolytic enzyme is selected from the group consisting of papain, collagenase, serratiopeptidase, heparanase, DNase, elastase, bromelain, bradykinase, Clostridium peptidase, enzymes expressed by Lactobacillus acidophilus, enzymes expressed by the Aspergillus genus, alliinase, and fibrinolysin.

51. The method of claim 48, wherein the vitamin is selected from retinaldehyde and retinoic acid and their precursors and natural or synthetic derivatives.

52. The method of claim 40, wherein the antidepressant compound is formulated in a substantially solid, gel or liquid preparation.

53. The method of claim 52, wherein the antidepressant compound is formulated in a preparation selected from the group consisting of creams, ointments, pomades, powders, plasters, impregnated membranes, solutions, emulsions, vesicular dispersions, lotions, gels, sprays and suspensions.

54. The method of claim 40, wherein the antidepressant compound is formulated in a substantially solid or gel preparation and is administered in an amount in a range from 100 mg/kg to 100 g/kg.

55. The method of claim 40, wherein the antidepressant compound is formulated in a substantially liquid preparation and is administered in an amount in a range from 100 mg/L to 100 g/L.

56. The method of claim 47, wherein the antidepressant compound is formulated in a substantially solid or gel preparation in combination with at least one proteolytic enzyme, the proteolytic enzyme being administered in an amount in the range from 1 mg/kg to 1 g/kg.

57. The method of claim 47, wherein the antidepressant compound is formulated in a substantially liquid preparation in combination with at least one proteolytic enzyme, the proteolytic enzyme in an amount in a range from 1 mg/L to 1 g/L.

58. The method of claim 48, wherein the antidepressant compound is formulated in a substantially solid or gel preparation in combination with at least one vitamin, the at least one vitamin in an amount in a range from 0.001 mg/kg to 10 g/kg.

59. The method of claim 48, wherein the antidepressant compound is formulated in a substantially liquid preparation in combination with at least one vitamin, the at least one vitamin being administered in an amount in a range from 0.001 mg/L to 10 g/L.

60. The method of claim 40, wherein the antidepressant compound is formulated in a pharmaceutical composition, a cosmetic composition, a medical device, or a culture medium.

61. A composition comprising
an antidepressant compound in combination with a further active ingredient selected from a proteolytic enzyme and a vitamin, in a physiologically acceptable vehicle or diluent
the composition being effective to obtain at least one of:
stimulation of regeneration of the integumentary system of a mammal;
stimulation of at least one of growth, original trophism, and original pigmentation of cutaneous appendages corresponding to the integumentary system of the mammal;
regeneration of damaged skin tissue of the mammal; and
treating of at least one of alopecia, effluvium, and defluvium of the mammal.

62. The composition of claim 61, comprising the antidepressant compound, the proteolytic enzyme and the vitamin.

63. The composition of claim 61, wherein the antidepressant compound is a selective serotonin reuptake inhibitor (SSRI).

64. The composition of claim 63, wherein the antidepressant compound is selected from the group consisting of hypericum, paroxetine, fluoxetine, fluvoxamine, amitriptyline, desipramine, chlorimipramine, imipramine, nortriptyline and venlafaxine.

65. The composition of claim 64, wherein the antidepressant compound is paroxetine.

66. The composition of claim 61, wherein the proteolytic enzyme is selected from the group consisting of papain, collagenase, serratiopeptidase, heparanase, DNase, elastase, bromelain, bradykinase, Clostridium peptidase, enzymes expressed by Lactobacillus acidophilus, enzymes expressed by the Aspergillus genus, alliinase, and fibrinolysin.

67. The composition of claim 61, wherein the vitamin is retinaldehyde or retinoic acid.

68. The composition of claim 61, wherein the composition is substantially solid or gel or liquid.

69. The composition of claim 61, wherein the composition is in form of a cream, ointment, pomade, powder, plaster, impregnated membrane, solution, emulsion, vesicular dispersion, lotion, gel, spray or suspension.

70. The composition of claim 61, wherein the composition is in a substantially solid or gel form, and in which the antidepressant compound is present in an amount in the range from 100 mg/kg to 100 g/kg.

71. The composition of claim 61, wherein the composition is in a substantially liquid form, and in which the antidepressant compound is present in an amount in the range from 100 mg/L to 100 g/L.

72. The composition of claim 70, comprising the proteolytic enzyme in an amount in the range from 1 mg/kg to 1 g/kg.

73. The composition of claim 71, comprising the proteolytic enzyme in an amount in the range from 1 mg/L to 1 g/L.

74. The composition of claim 70, comprising the vitamin in an amount in the range from 0.001 mg/kg to 10 g/kg.

75. The composition of claim 71, comprising the vitamin in an amount in the range from 0.001 mg/L to 10 g/L.

76. The composition of claim 61, wherein the composition is a pharmaceutical or cosmetic composition, a medical device, or a culture medium.

77. The composition of claim 61, comprising one or more further ingredients selected from the group comprising of vitamins, vitamin factors, glucosaminoglycans, saccharides, triterpene acids or heterosides, peptides, amino acids, supplements, corticosteroids, and anticholinergics.
